(19) United States
(12) Patent Application Publication
(10) Pub. No.: US 2023/0123594 A1
(43) Pub. Date: Apr. 20, 2023

(54) ANTIBIOTIC SUSCEPTIBILITY TEST

(71) Applicant: Fundamental Solutions Corporation, Easton, PA (US)

(72) Inventor: Joseph D. Kittle, JR., Easton, PA (US)

(21) Appl. No.: 17/965,207

(22) Filed: Oct. 13, 2022

Related U.S. Application Data
(60) Provisional application No. 63/256,059, filed on Oct. 15, 2021.

(51) Int. Cl. C12Q 1/18; G01N 21/64; G01N 33/569

(52) U.S. Cl. CPC C12Q 1/18; G01N 21/6428; G01N 33/56911; G01N 2021/6439; G01N 2333/245

(57) ABSTRACT

A method for determining the susceptibility of a bacteria to an antibiotic, comprising transferring one portion of a sample containing living bacterial cells into a bacterial growth medium to create a control sample; transferring another portion of the sample into a bacterial growth medium to which a predetermined amount of antibiotic or predetermined amount of a library of antibiotics has been added to create a test sample; adding an alkyne-modified non-canonical amino acid to both the control sample and test sample during bacterial growth, wherein the alkyne-modified non-canonical amino acid incorporates into surface proteins, internal proteins, or both; reacting the alkyne-containing proteins with an azide-modified detection molecule using click-chemistry to label the cells; detecting the labeled cells using a method that generates a detectable signal; and comparing the signal generated by the control sample to the signal generated by the test sample, wherein a decrease in detectable signal between the control sample and the test sample is indicative of susceptibility of the living bacteria to the predetermined antibiotic or predetermined library of antibiotics.

This patent application claims the benefit of U.S. Provisional Patent Application Ser. No. 63/256,059 filed on Oct. 15, 2021, and entitled “Antibiotic Susceptibility Test”, the disclosure of which is hereby incorporated by reference herein in its entirety and made part of the present U.S. utility patent application for all purposes.

### BACKGROUND

The disclosed inventive subject matter relates in general to diagnostic systems, devices, and methods for use in infectious disease, and more specifically to a rapid antimicrobial or antibiotic susceptibility test for directly detecting bacterial susceptibility to various antibiotics.

Selecting a proper antibiotic to treat a bacterial infection is typically accomplished through either polymerase chain reaction (PCR) identification of the bacteria and choosing a standard course of antibiotics or by directly testing antibiotic susceptibility to determine which antimicrobials will inhibit the growth of the bacteria causing a specific infection. Bacteria may be identified with PCR; however, PCR does not directly confirm the susceptibility of the identified bacteria to a standard treatment regimen. Ineffective or incomplete treatment with antibiotics can lead to the development of antibiotic resistant strains of bacteria, which is a widely recognized problem in healthcare. Direct antimicrobial susceptibility testing or antibiotic susceptibility testing (AST) may suggest a more successful treatment regimen, but such testing is much slower and more labor-intensive. Accordingly, there is an ongoing need for a high-throughput, rapid, reliable, and easy to use assay for directly determining bacterial susceptibility to a library of antibiotics.

### SUMMARY

The following provides a summary of certain example implementations of the disclosed inventive subject matter. This summary is not an extensive overview and is not intended to identify key or critical aspects or elements of the disclosed inventive subject matter or to delineate its scope. However, it is to be understood that the use of indefinite articles in the language used to describe and claim the disclosed inventive subject matter is not intended in any way to limit the described inventive subject matter. Rather the use of “a” or “an” should be interpreted to mean “at least one” or “one or more”.

One implementation of the disclosed technology provides a method for determining the susceptibility of a bacteria to an antibiotic, comprising obtaining a patient sample containing living bacterial cells; transferring one portion of the patient sample into a bacterial growth medium to create a control sample; transferring another portion of the patient sample in a bacterial grow medium to which a predetermined amount of antibiotic or predetermined amount of a library of antibiotics has been added to create a test sample; adding an alkyne-modified non-canonical amino acid to the bacterial growth medium of both the control sample and test sample during bacterial growth, wherein the alkyne-modified non-canonical amino acid incorporates into surface and/or internal proteins of the growing bacteria; reacting the alkyne-containing proteins with an azide-modified detection molecule using click-chemistry to label the living bacteria cells in a detectable manner; detecting the labeled bacterial cells using a method that generates a detectable signal; and comparing the signal generated by the control sample to the signal generated by the test sample, wherein a decrease in detectable signal between the control sample and the test sample is indicative of susceptibility of the living bacteria to the predetermined antibiotic or predetermined library of antibiotics.

The patient sample may be a biological sample derived from a bodily fluid or other bodily source. The antibiotic may be chloramphenicol or any other antibiotic or combination of antibiotics. The non-canonical amino acid may be azide-modified rather than alkyne-modified and the detection molecule may be alkyne-modified rather than azide-modified. The alkyne-modified non-canonical amino acid may be L-Homopropargylglycine. The azide-modified detection molecule may be a biotinylated ligand. The azide-modified detection molecule may be a fluorogenic azide probe. The method that generates a detectable signal may be fluorescence-based. The method that generates a detectable signal may be enzyme-linked immunosorbent assay (ELISA)-based. The method that generates a detectable signal may be P5G7 cell-based or P2D8 cell-based. The method that generates a detectable signal may be dot blot-based or microscopy-based. The signal may be quantifiable, and a predetermined amount of signal may be indicative of a minimal inhibitory concentration (minimal effective amount) of antibiotic. The method may be high-throughput method executed on a multi-well plate or microplate, wherein the type of multi-well plate or microplate may include filter plates, and wherein more than one type of antibiotic may be tested on the multi-well plate or microplate.

Another implementation of the disclosed technology provides a method for determining the susceptibility of a bacteria to an antibiotic, comprising obtaining a patient sample containing living bacterial cells, wherein the patient sample is a biological sample derived from a bodily fluid or other bodily source; transferring one portion of the patient sample into a bacterial growth medium to create a control sample; transferring another portion of the patient sample in a bacterial grow medium to which a predetermined amount of antibiotic or predetermined amount of a library of antibiotics has been added to create a test sample; adding an alkyne-modified non-canonical amino acid to the bacterial growth medium of both the control sample and test sample during bacterial growth, wherein the alkyne-modified non-canonical amino acid incorporates into surface and/or internal proteins of the growing bacteria, and wherein the alkyne-modified non-canonical amino acid is L-Homopropargylglycine; reacting the alkyne-containing proteins with an azide-modified detection molecule using click-chemistry to label the living bacteria cells in a detectable manner; detecting the labeled bacterial cells using a method that generates a detectable signal; and comparing the signal generated by the control sample to the signal generated by the test sample, wherein a decrease in detectable signal between the control sample and the test sample is indicative of susceptibility of the living bacteria to the predetermined antibiotic or predetermined library of antibiotics.

The antibiotic may be chloramphenicol or any other antibiotic or combination of antibiotics. The non-canonical amino acid may be azide-modified rather than alkyne-modified and the detection molecule may be alkyne-modified rather than azide-modified. The azide-modified detection molecule may be a biotinylated ligand. The azide-modified detection molecule may be a fluorogenic azide probe. The method that generates a detectable signal may be fluorescence-based. The method that generates a detectable signal may be enzyme-linked immunosorbent assay (ELISA)-based. The method that generates a detectable signal may be P5G7 cell-based or P2D8 cell-based. The method that generates a detectable signal may be dot blot-based or microscopy-based. The signal may be quantifiable, and a predetermined amount of signal may be indicative of a minimal inhibitory concentration (minimal effective amount) of antibiotic. The method may be high-throughput method executed on a multi-well plate or microplate, wherein the type of multi-well plate or microplate may include filter plates, and wherein more than one type of antibiotic may be tested on the multi-well plate or microplate.

Still another implementation of the disclosed technology provides a method for determining the susceptibility of a bacteria to an antibiotic, comprising obtaining a patient sample containing living bacterial cells, wherein the patient sample is a biological sample derived from a bodily fluid or other bodily source; transferring one portion of the patient sample into a bacterial growth medium to create a control sample; transferring another portion of the patient sample in a bacterial growth medium to which a predetermined amount of antibiotic or predetermined amount of a library of antibiotics has been added to create a test sample; adding an alkyne-modified non-canonical amino acid to the bacterial growth medium of both the control sample and test sample during bacterial growth, wherein the alkyne-modified non-canonical amino acid incorporates into surface and/or internal proteins of the growing bacteria, and wherein the alkyne-modified non-canonical amino acid is L-Homopropargylglycine; reacting the alkyne-containing proteins with an azide-modified detection molecule using click-chemistry to label the living bacteria cells in a detectable manner, wherein the azide-modified detection molecule is a biotinylated ligand or a fluorogenic azide probe; detecting the labeled bacterial cells using a method that generates a detectable signal; and comparing the signal generated by the control sample to the signal generated by the test sample, wherein a decrease in detectable signal between the control sample and the test sample is indicative of susceptibility of the living bacteria to the predetermined antibiotic or predetermined library of antibiotics.

The antibiotic may be chloramphenicol or any other antibiotic or combination of antibiotics. The non-canonical amino acid may be azide-modified rather than alkyne-modified and the detection molecule may be alkyne-modified rather than azide-modified. The method that generates a detectable signal may be fluorescence-based. The method that generates a detectable signal may be enzyme-linked immunosorbent assay (ELISA)-based. The method that generates a detectable signal may be P5G7 cell-based or P2D8 cell-based. The method that generates a detectable signal may be dot blot-based or microscopy-based. The signal may be quantifiable, and a predetermined amount of signal may be indicative of a minimal inhibitory concentration (minimal effective amount) of antibiotic. The method may be high-throughput method executed on a multi-well plate or microplate, wherein the type of multi-well plate or microplate may include filter plates, and wherein more than one type of antibiotic may be tested on the multi-well plate or microplate.

It should be appreciated that all combinations of the foregoing concepts and additional concepts discussed in greater detail below (provided such concepts are not mutually inconsistent) are contemplated as being part of the inventive subject matter disclosed herein and may be implemented to achieve the benefits as described herein. Additional features and aspects of the disclosed system, devices, and methods will become apparent to those of ordinary skill in the art upon reading and understanding the following detailed description of the example implementations. As will be appreciated by the skilled artisan, further implementations are possible without departing from the scope and spirit of what is disclosed herein. Accordingly, the drawings and associated descriptions are to be regarded as illustrative and not restrictive in nature.

### BRIEF DESCRIPTION OF THE DRAWINGS

The accompanying drawings, which are incorporated into and form a part of the specification, schematically illustrate one or more example implementations of the disclosed inventive subject matter and, together with the general description given above and detailed description given below, serve to explain the principles of the disclosed subject matter, and wherein:

FIG. 1 depicts a click chemistry reaction scheme using Cu(I)-catalyzed azide— alkyne cycloaddition (CuAAC);

FIG. 2 depicts the general workflow of the disclosed antibiotic susceptibility test (AST);

FIG. 3 is a series of images depicting a dot blot for biotinylation detection of bacteria;

FIGS. 4A-4C are a series of images depicting biotinylation detection of bacteria after non-canonical amino acid incorporation;

FIGS. 5A-5C are a series of images depicting biotinylation detection of bacteria after treatment with chloramphenicol;

FIG. 6 depicts an ELISA detection method for the disclosed AST; and

FIG. 7 depicts the AST responses of E. coli to 100 μg/mL chloramphenicol and 200 μg/mL nitrofurantoin.

### DETAILED DESCRIPTION

Example implementations are now described with reference to the Figures. Reference numerals are used throughout the detailed description to refer to the various elements and structures. Although the following detailed description contains many specifics for the purposes of illustration, a person of ordinary skill in the art will appreciate that many variations and alterations to the following details are within the scope of the disclosed inventive subject matter. Accordingly, the following implementations are set forth without any loss of generality to, and without imposing limitations upon, the claimed subject matter.

With reference to the Figures, the disclosed AST (which may be referred to as “BLAST”) determines antibiotic susceptibility by detecting changes in the number or amount of living bacteria after the bacteria have been incubated with a predetermined library of antibiotics. This assay exploits the fact that living bacteria have a significantly faster metabolism and protein production than dead or dying bacteria and will take up amino acids and incorporate them into newly formed proteins at a much faster rate. The assay replaces methionine in a bacterial media with a non-canonical amino acid (ncAA), which includes a specific reactive group, thereby enabling specific detection of living bacteria (see, for example, Sherratt et al. Rapid Screening and Identification of Living Pathogenic Organisms via Optimized Bioorthogonal Non-canonical Amino Acid Tagging, Cell Chemical Biology 24, 1048-1055 (2017), which is incorporated by reference herein in its entirety). In one example implementation, the assay includes three important interactions for successfully detecting living bacteria: (i) bacterial incorporation of the ncAA, (ii) a click-chemistry type reaction between the reactive group of the ncAA and a labeled (e.g., biotinylated) ligand having an azide group, and (iii) detection of the newly biotinylated ligand using a predetermined type of cell, such as for example, a CytoSPAR™ P5G7 cell (see Kittle et al., Development of a Surface Programmable Activation Receptor system (SPAR): A living cell biosensor for rapid pathogen detection, bioRxiv 687426; doi: https://doi.org/10.1101/687426, which is incorporated by reference herein in its entirety for all purposes).

L-Homopropargylglycine (HPG) is an alkyne modified ncAA that mimics methionine during protein production. When HPG is present in bacterial growth media, bacteria growing in the media will incorporate HPG into newly synthesized proteins. Alkyne groups are not naturally found in bacterial cells and serve as a specific reactive group for bacteria undergoing active protein synthesis. HPG has been detected in bacteria after just a 30-minute incubation period, making the entire process comparatively fast.

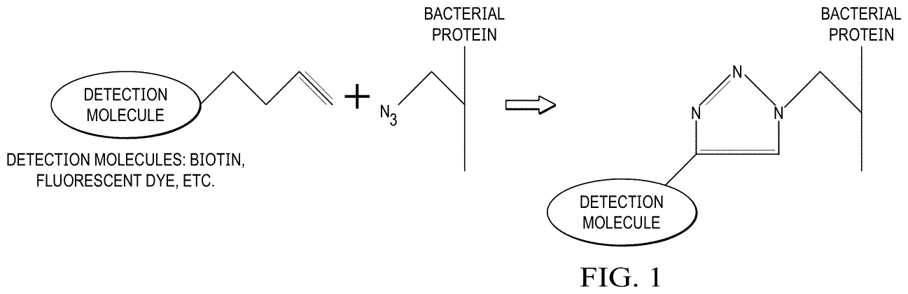

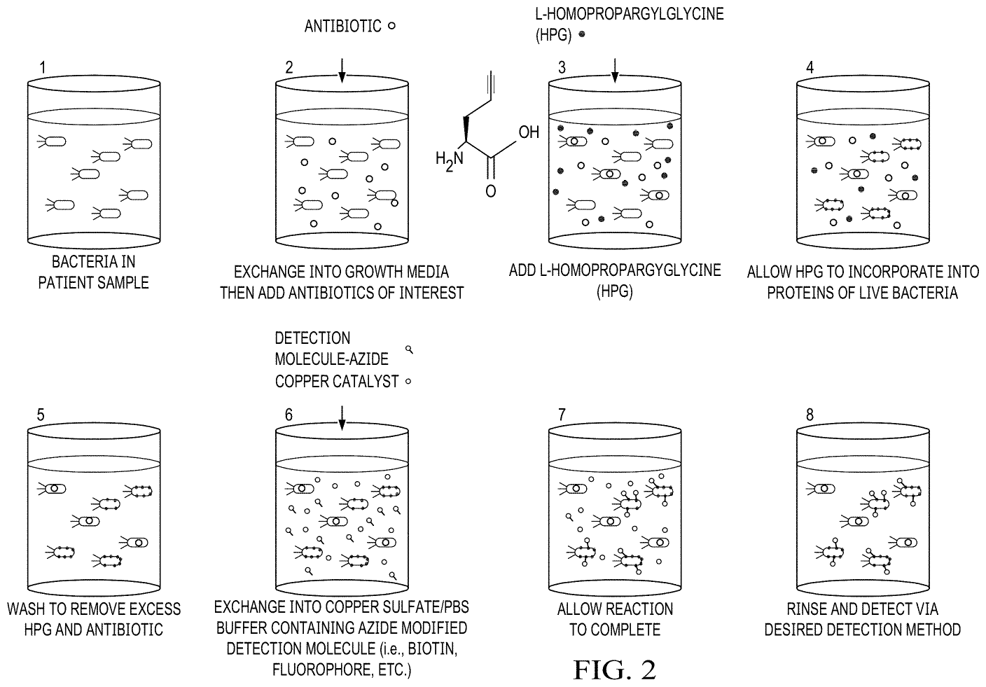

The alkyne group is one component of the copper catalyzed alkyne-azide cycloaddition (CuAAC), more commonly known as click chemistry (see, for example, Atwal et al., Clickable methionine as a universal probe for labelling intracellular bacteria, Journal of Microbiological Methods 169 (2020) 105182; and Li et al., Fluorogenic “click” reaction for labeling and detection of DNA in proliferating cells, BioTechniques 49:525-527 (July 2010), both of which are incorporated by reference herein in their entirety for all purposes). When a ligand with an azide group encounters an alkyne group, the reaction creates an irreversible ring structure (see FIG. 1). After bacteria uptake of HPG, alkyne groups can be found in any protein that includes a methionine amino acid, including internal proteins and surface proteins. Because the CuAAC reaction specifically labels living bacteria at their surface, in some implementations, cell lysis may be eliminated as a necessary aspect of the assay. However, in other implementations, cell lysis may be employed for assay optimization and for increasing the amount of signal generated by the assay. A general workflow for the disclosed assay is shown in FIG. 2. The assay has been demonstrated to detect antibiotic susceptibility in as few as five (5) hours, depending on patient sample.

Multiple detection methods can be used with the disclosed assay, including fluorescence, cells, blotting, and ELISA-based methods depending on which detection molecule is chosen. To detect antibiotic susceptibility, the signal produced from a control sample (no antibiotic treatment) is compared with samples treated with antibiotics, thereby detecting changes in bacterial protein production that correlate to antibiotic susceptibility.

### Materials and Methods

TABLE 1
Reagents, Consumables, and Equipment.
Reagents & Consumables

| Item | Vendor | Cat # |
|---|---|---|
| Nunc MaxiSorp 96 Well ELISA | ThermoFisher | 44-2404-21 |
| Plate | | |
| Streptavidin | MP Bio | 08623001 |
| E. coli UTI Derived | ATCC | ATCC 25922 |
| S. saprophyticus UTI Derived | ATCC | ATCC 15305 |
| Streptococcus pyogenes | ATCC | ATCC 19615 |
| Pseudomonas aeruginosa | ATCC | ATCC 27853 |
| Chloramphenicol, ≥98% (HPLC) | Sigma Aldrich | C0378-25G |
| Nitrofurantoin (98-102%) | Sigma | N7878-25G |
| Magnesium Sulfate, Anhydrous, | Sigma Aldrich | MX0075-1 |
| powder Meets Reagent | | |
| Specifications for testing | | |
| USP/NF monographs GR | | |
| α-D-Glucose, anhydrous, 96% | Sigma Aldrich | 158968-100G |
| MEM Vitamin Solution (100×), | Sigma Aldrich | M6895-100ML |
| sterile-filtered, BioReagent, | | |
| suitable for cell culture | | |
| L-lysine | Sigma Aldrich | L8662 |
| L-threonine | Sigma Aldrich | T8441 |
| L-phenylalanine | Sigma Aldrich | P5482 |
| L-isoleucine | Sigma Aldrich | I7403 |
| L-valine | Sigma Aldrich | V0513 |
| L-methionine | Sigma Aldrich | M5308 |
| Copper(II) sulfate pentahydrate, | Sigma Aldrich | 209198-100G |
| ACS reagent, ≥98.0% | | |
| L-Histidine | Sigma Aldrich | H8000 |
| (+)-Sodium L-ascorbate, | Sigma Aldrich | A7631 |
| crystalline, ≥98% | | |
| Phosphate Buffered Saline (PBS) | VWR | VWRVE703-1L |
| 20X, Ultra Pure Grade | | |
| Mouse IgG2A anti-biotin (Biotin | ThermoFisher | 200-301-098 |
| Monoclonal Antibody (4C7.D5)) | | |
| SuperBlock ™ T20 (PBS) | ThermoFisher | 37516 |
| Blocking Buffer | | |
| NHS-Biotin | Click Chemistry | B102-100 |
| | Tools | |
| Biotin-Azide Plus | Click Chemistry | 1167-5 |
| | Tools | |
| L-Homopropargylglycine (HPG) | Click Chemistry | 1067-25 |
| | Tools | |
| Gibco ™ LB Broth | Gibco | 10855-021 |
| Gibco ™ M9 Minimal Salts (2X) | Gibco | A1374401 |
| Alfa Aesar ™ L-Leucine, Cell | Alfa Aesar | AAJ6282422 |
| Culture Reagent | | |
| Invitrogen ™ eBioscience ™ | Invitrogen | 50-112-8941 |
| Avidin HRP | | |
| Alfa Aesar ™ Calcium chloride, | Alfa Aesar | AAL1319130 |
| dried, powder, 97% | | |
| Super Signal West Pico Plus | Thermo Scientific | 34580 |
| Chemiluminescent | | |
| Substrate | | |
| CalFluor 488 Azide | Click Chemistry | 1369-1 |
| | Tools | |
| Immun-Blot PVDF Membrane | Bio-Rad | 1620177 |
| 20X PBS Tween-20 | Thermo Scientific | 28352 |
| AcroPrep ™ Advance Plate, | PALL | 8019 |
| Short Tip; 0.2 μm; Supor | | |
| (PES) Membrane | | |
| 96 well receiver plate | Agilent | 204600-100 |

Equipment

| Item | Vendor |
|---|---|
| Li-Cor C-Digit Blot Scanner | Li-Cor |
| Victor X5 Microplate Reader | Perkin Elmer |
| SpectraMax L Luminometer | Molecular |
| | Devices |

An example protocol for performing the disclosed assay includes performing the following assay methodology. Culture and resuspension volumes are held constant throughout the method (i.e., if a culture was 1 mL, then it was re-suspended in 1 mL of appropriate media in further steps). A high throughput microplate method is an aspect of the disclosed assay and is outlined below.

Media and Buffer Preparation
1. Prepare supplemented M9 media by adding the following supplements to the indicated final concentrations: 100 μM CaCl, 1 mM MgSO4, 16.65 mM Glucose, and 1× MEM Vitamin mixture.
2. Prepare methionine inhibition growth media (MIGM) by adding the following amino acids to supplemented M9 media at the indicated final concentration: L-lysine (100 μg/mL), L-threonine (100 μg/mL), L-phenylalanine (100 μg/mL), L-isoleucine (50 μg/mL), L-leucine (50 μg/mL), and L-valine (50 μg/mL).
3. Prepare separate 2.5 mg/mL stock solutions of each L-homopropargylglycine and L-methionine in supplemented M9 media. This is a 50× stock solution of each amino acid to be added to MIGM.
4. Prepare click-chemistry buffer (CCB) using 100 μM CuSO4, 200 μM L-histidine, 2 mM sodium ascorbate, and either 100 μM of biotin-azide (CCB-Biotin) or 488-azide (CCB-488) in PBS (pH 7.5).

Bacteria Culture and Sample Preparation
1. Patient sample preparation is based on an E. coli preparation.
2. Plate E. coli on agar plates overnight at 37° C.
3. Select single colonies and inoculate LB overnight at 37° C. with gentle agitation.
4. Add LB culture to supplemented M9 media (1:4, LB:M9 volume) and incubate at 37° C. with gentle agitation for 2 hours.

Alkyne Labeling of Living Bacteria
1. This method incubates bacteria under 6 different experimental conditions as seen in Table 2, below.
   a. A typical assay will treat a patient sample with HPG/no detection azide (negative control), HPG/detection azide (positive control), and HPG/drugs of choice/detection Azide.
2. Pellet LB/M9 culture and reconstitute in supplemented M9 media.
3. Split culture into three equal volumes, pellet, and reconstitute in MIGM.
4. Prepare drug stock solution in 200 proof ethanol.
   a. For this study, the drug chloramphenicol was used as a model antibiotic. Due to its low water solubility, a 50 mg/mL stock solution was made in absolute ethanol and diluted to 50 μg/mL in MIGM. This results in a 0.1% ethanol solution.
5. Add control ethanol to two cultures and drug stock solution to one culture.
6. Incubate for 30 minutes at 37° C. with gentle agitation.
7. Add stock solutions of HPG or Methionine to the appropriate cultures as outlined in Table 2.
8. Incubate for 2 additional hours under the same conditions.
9. Pellet bacteria and wash 3 times with PBS.

TABLE 2
Experimental Conditions

| | HPG/Chloramphenicol | HPG Only | Methionine Only |
|---|---|---|---|
| Biotin-Azide | X | X | X |
| 488-Azide | X | X | X |

Biotin and Fluorescent Labeling of Alkyne Modified Bacteria
1. Resuspend Pellet in PBS and split each culture in two equal volumes, one will receive 488-azide while the other will receive biotin-azide.
2. Pellet the bacteria and resuspend in CCB-Biotin buffer or CCB-488 buffer as appropriate.
3. Incubate at 37° C. with gentle agitation for 30 minutes.
4. Pellet bacteria and rinse 3 times in PBS.

Fluorescent and Biotinylation Detection Methods
1. While the bacteria are undergoing the click-chemistry reaction, allow P5G7 cells to thaw at room temp for 30 minutes.
2. Add anti-biotin antibody (final concentration of 5 μg/mL) to the cells and incubate for a further 30 minutes at room temperature.
3. Resuspend bacteria pellet in PBS and set aside 300 μL for fluorescent detection.
4. Pellet bacteria and resuspend in DMEM.
5. While the bacteria are pelleting, prepare a black plate with 3 replicates of 100 of each experimental condition. Read these in a top-read, fluorescent plate reader. (Ex/Em: 500/521 optimal, 1 second integration time).
6. Add 30 μL of each experimental condition mixture in duplicate to a white plate.
7. Prepare a luminometer to read the plate using a 1 second integration time and a kinetic read over 20 minutes.
8. Pipette 90 μL of the P5G7 cells/antibody mixture in to each well and read immediately.

Dot Blot Detection
1. Prepare a PVDF membrane by soaking it in methanol for at least 5 minutes.
2. Remove excess methanol and immediately add 2 μL of each condition mixture to the activated membrane.
3. Allow the membrane to dry completely.
4. Reactivate the membrane with methanol for another 5 minutes.
5. Remove excess methanol and cover the membrane in blocking buffer for 1 hour at room temperature with gentle rocking.
6. Cover membrane in Avidin-HRP solution (1/2000 dilution in PBS-T) for 1 hour at room temperature with gentle rocking.
7. Rinse membrane with PBS-T 3 times (10 minutes each).
8. Soak membrane in ECL for 5 minutes and image blot.

ELISA Based Detection
1. Add 200 μL of streptavidin coating solution (5 μg/mL in PBS) to each well of a high adsorption 96-well microplate, cover, and incubate overnight at 4° C.
2. Wash the plate once with wash buffer (PBS-T, PBS with 0.05% tween-20).
3. Place bacteria from the AST in the wells of the streptavidin coated plate and allow to adsorb for 1 hour at room temperature.
4. Rinse the plate 3 times with wash buffer.
5. Block the plate with blocking buffer for 1 hour at room temperature.
6. Dilute Avidin-HRP 1/500 in blocking buffer and then add to the wells. Incubate the microplate at room temperature for 1 hour.
7. Rinse the plate 3 times in wash buffer.
8. Place 100 μL of TMB solution in each well and allow to incubate at room temperature for 30 minutes.
9. Place 100 μL of Stop solution (1N HCL) in each well.
10. Measure absorbance at 450 nm for each well.

Data Analysis
1. For P5G7 cell-based detection, take the area under the curve from 180 s to 540 s by summing all RLU readings between these time points and then multiplying by the time between readings. Compare AUC values between groups to determine detection of biotinylation.
2. For fluorescent detection, compare the RFU readings between each group to determine detection of the fluorescent dye.
3. Ultimately, antibiotic susceptibility is detected when the control conditions (HPG with azide-biotin or azide-488) present a larger signal than those conditions which contain drug.

High Throughput Microplate Method
A. A filter plate method may be used as a high throughput method.
B. All steps are done on the plate, including bacteria growth, but working volumes are reduced to 300 μL.
C. Patient samples are added to the plate in multiple wells and the media is filtered through the plate rather than pelleting the bacteria:
1. Determine the OD600 of the overnight culture using a spectrophotometer.
2. Use the overnight culture to inoculate supplemented M9 medium at a ratio of 1:32 (overnight LB culture: supplemented M9 medium). Add 0.3 mL of the inoculated supplemented M9 medium into each well of the 96 well filter plate (skip wells when using translucent plates).
3. Incubate the filter plate at 37° C. with shaking at 250 rpm for 2 hours
4. Filter the plate to remove the media and wash once using PBS. Resuspend the pellet in MIGM medium containing different antibiotics as described in Table 3.
5. Incubate the filter plate at 37° C. with shaking at 250 rpm for 30 minutes.
6. Filter the plate to remove the media and then resuspend the pellet in MIGM medium containing different antibiotics and respective amino acids as designed in Table 3.
   a. Prepare 16 (0.3 mL/well) culture conditions as follows:
   b. 4 cultures in MIGM containing L-methionine (50 μg/mL)
   c. 4 cultures in MIGM containing L-Homopropargylglycine (50 μg/mL)
   d. 4 cultures in MIGM containing L-Homopropargylglycine (50 μg/mL); Chloramphenicol (100 μg/mL)
   e. 4 cultures in MIGM containing L-Homopropargylglycine (50 μg/mL); Nitrofurantoin (200 μg/mL)
7. Incubate the plate for 2 hours at 37° C. with shaking at 250 rpm.
8. Filter the plate and rinse 3 times with PBS. Reconstitute the pellets in Click-chemistry buffer (CCB) containing 488-azide (50μM) or buffer alone (Table 3).
   a. The test conditions are:
      i. L-methionine/488-azide
      ii. L-methionine alone
      iii. HPG/488-azide
      iv. HPG/488-azide/Chloramphenicol
      v. HPG/488-azide/Nitrofurantoin
      vi. HPG alone
9. Mix thoroughly by pipetting and then incubate the plate at 37° C. for 30 minutes.
10. Rinse the plate 3 times with PBS and remove all the buffer.
11. Read the signal in a fluorescent plate reader at 484 excitation and 524 emission.

### Results

FIG. 1 depicts a click chemistry reaction scheme using Cu(I)-catalyzed azide— alkyne cycloaddition (CuAAC). FIG. 2 depicts the general workflow of the disclosed antibiotic susceptibility test (AST).

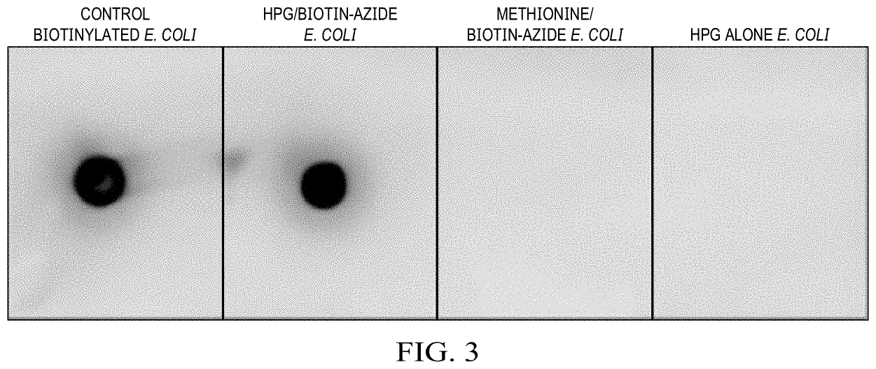

FIG. 3 is a series of images depicting a dot blot for biotinylation detection of bacteria, wherein 2 μL of bacteria from the disclosed AST were placed on a PVDF membrane and detected with avidin-HRP/ECL. Control biotinylated bacteria were produced by reacting E. coli with NHS-Biotin for 30 minutes at room temperature. A dot blot is a facile detection method for biotinylation using avidin-HRP. FIG. 3 shows the dot blot with only the control bacteria (biotinylated E. coli labeled through an NHS-Biotin reaction) and bacteria which received HPG, thereby indicating that the click-chemistry reaction is successful under these conditions.

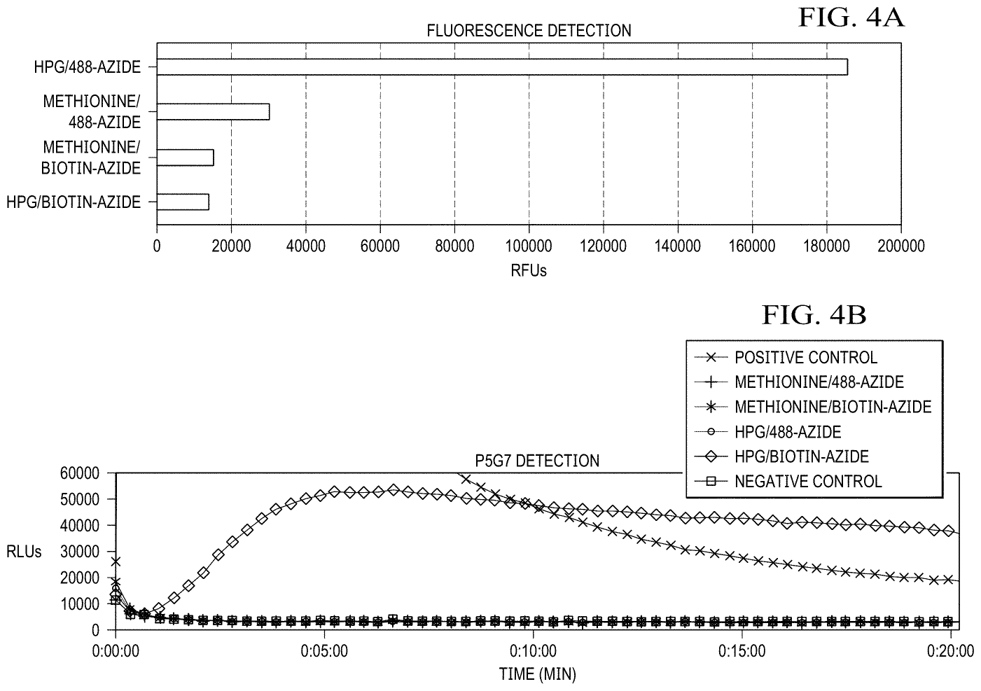

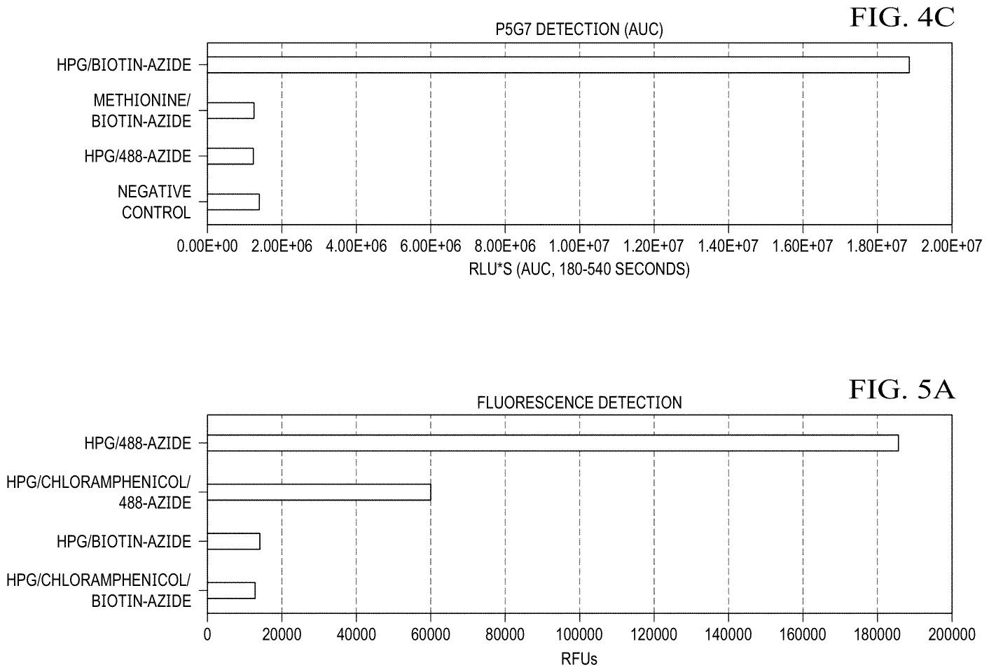

To better detect biotinylated bacteria, a fluorescent and P5G7 cell luminescent detection method was developed. FIGS. 4A-4C are a series of images depicting biotinylation detection of bacteria after ncAA incorporation. Shown are fluorescent (FIG. 4A) or P5G7 based detection (FIGS. 4B-4C) of bacteria that received either HPG or methionine control and an azide linked detection molecule. Fluorescence was measured on a Victor X5 fluorescent plate reader using a fluorescein filter set. FIG. 4B represents a kinetic read of luminescence over a 20-minute period. The positive control peaked at 2.8 million RLUs but is not shown in the Figures. FIG. 4C is the area under the curve (AUC) calculated from the data in the middle panel. FIGS. 4A-4C show the success of both methods at specifically detecting bacteria which have incorporated the ncAA, HPG. Bacteria which received methionine and 488-azide did have higher fluorescent signal than those which did not receive any fluorescent-azide, suggesting that some off-target reaction is occurring. However, this signal is small compared to the positive signal from the HPG/488-azide group.

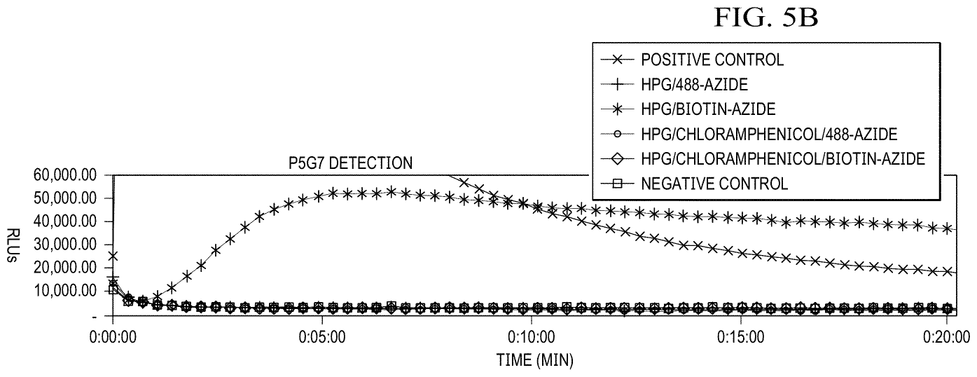

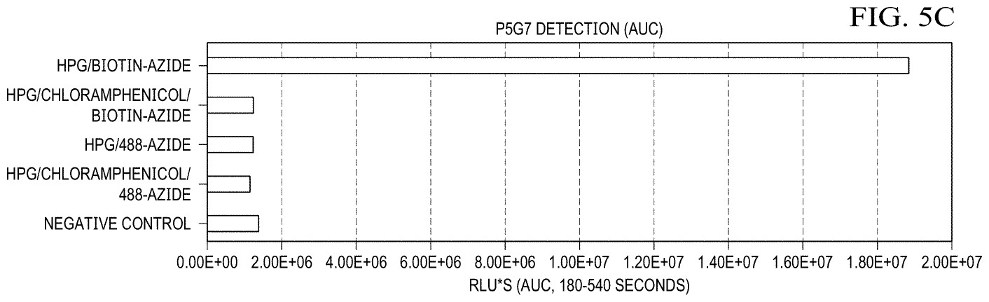

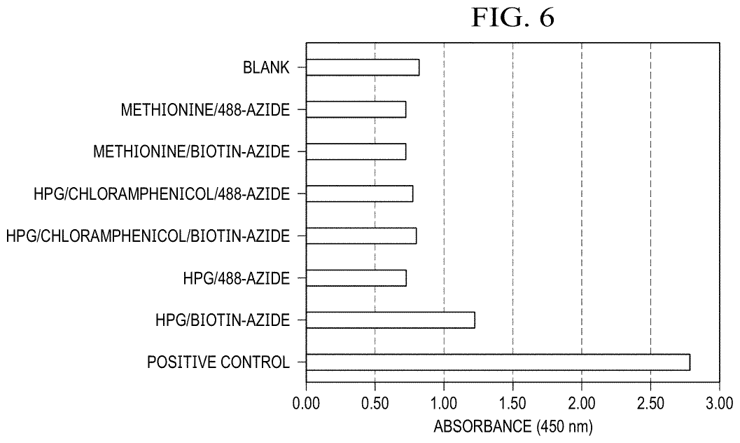

Chloramphenicol was chosen as a model antibiotic because it directly inhibits protein synthesis in bacteria. FIGS. 5A-5C are a series of images depicting biotinylation detection of bacteria after treatment with chloramphenicol. Shown are fluorescent (FIG. 5A) or P5G7 cell-based detection (FIGS. 5B-5C) of bacteria that received HPG, either chloramphenicol or control ethanol, and an azide-linked detection molecule. Fluorescence was measured on the Victor X5 fluorescent plate reader using a fluorescein filter set. FIG. 5B represents a kinetic read of luminescence over a 20-minute period. The positive control peaked at 2.8 million RLUs but is not shown in the Figures. FIG. 5C is the area under the curve (AUC) calculated from the data in the middle panel. FIGS. 5A-5C illustrate that bacteria which receive antibiotic prior to incubation with HPG have reduced signal in both detection methods, thereby indicating that the disclosed assay is effective for detecting antibiotic susceptibility. The results of a modified sandwich ELISA method confirmed these findings. FIG. 6 depicts an ELISA detection method for the disclosed AST, wherein bacteria used in FIGS. 5A-5C were captured on a streptavidin coated ELISA plate and detected with avidin-HRP/TMB.

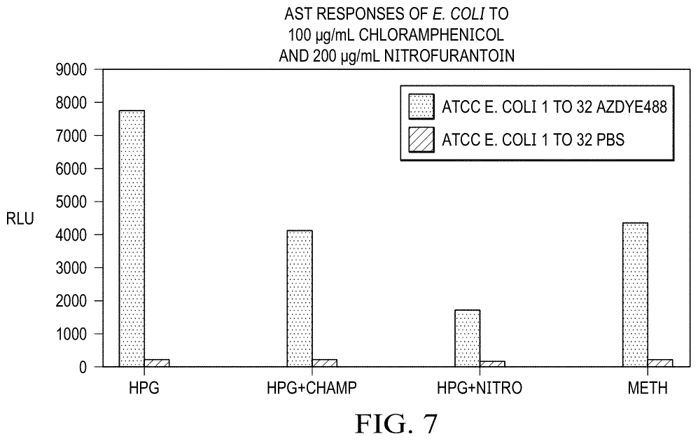

FIG. 7 provides results for AST responses of E. coli when treated with either 100 μg/mL chloramphenicol or 200 μg/mL nitrofurantoin. The starting overnight bacteria culture was diluted at 1:32 (overnight LB culture: supplemented M9 medium). The detection was performed using an azide-linked fluorescent tag. Bacteria samples were treated with either: HPG alone; HPG+chloramphenicol; HPG+nitrofurantoin; or methionine alone. Each sample was then reacted with an azide-linked detection molecule. Fluorescence was measured on Spectramax M2, Molecular Devices at 484 excitation and 524 emission. Results show that samples treated with either chloramphenicol or nitrofurantoin have reduced signal compared to non-treated samples (HPG alone). This is an indication that the disclosed assay is effective for detecting antibiotic susceptibility.

Advantages of the disclosed technology include the following: the assay does not require plating samples; the assay does not require bacterial replication; the assay is rapid and can be completed in about 2-5 hours; assay sensitivity is within an appropriate range for urinary tract infections (UTIs); the assay does not require strict identification of bacteria and works in polymicrobial cultures; and the assay can be easily customized, can be automated, and may include a numerical readout. The filter plate-based assay was entirely performed on the same plate from beginning of the process to the end therefore, the process can be easily automated.

The methods and results disclosed herein are intended to be examples, and as will be appreciated by one of ordinary skill in the art, various substitutions and modifications are possible. For example, in one implementation of the disclosed assay, an azide-containing non-canonical amino acid is used in the assay rather than an alkyne-modified non-canonical amino acid and this azide-containing non-canonical amino acid is reacted with an alkyne-modified or alkyne-labeled detector molecule rather than an azide-modified detection molecule. Azides of amino acids can be labeled with terminal alkyne or strained alkyne (e.g., DBCO)-tagged reporter molecules by way of a Cu(I)-catalyzed Alkyne-Azide (CUAAC) or Cu(I)-free strain-promoted Alkyne-Azide Click-Chemistry (SPAAC) reaction, respectively. Certain cell-permeable click-functionalized amino acids are randomly incorporated instead of methionine during translation and are therefore suitable for residue selective protein synthesis monitoring. In another example implementation of the disclosed assay, the method that generates a detectable signal utilizes a polypeptide protein tag such as, for example, a FLAG-tag, and a target detector molecule that is specific for the tagged polypeptide. In another example implementation, the method that generates a detectable signal is P2D8 cell-based when a target detection molecule that includes streptavidin is used (see for example, U.S. patent application Ser. No. 16/353,337, which is incorporated herein in its entirety for all purposes).

In some implementations of the disclosed assay, the growth medium contains a desired amino acid analog and simply allows the bacterial cells to metabolize or at least undergo protein synthesis. In some implementations, the growth medium contains only the amino-acid analog and a buffer. While alkyne-modified non-canonical amino acids can be incorporated into proteins of growing bacterial cells using the disclosed methods, the bacterial cells may also be lysed using an alkaline buffer, for example, to increase the detectable signal by including internal bacterial proteins in the detection method. Alternately, the cells may be permeabilized or fixed. Some implementations include the use of fluorogenic azide tags that increase in brightness (quantum yield) when reacted with the alkyne group on the labeled protein, thereby resulting in lower background from unreacted tag. Other implementations include the use of fluorogenic dyes as the detection molecule. See, for example, Beatty et al., Selective Dye-Labeling of Newly Synthesized Proteins in Bacterial Cells, J. Am. Chem. Soc. 127: 14150-14151 (2005); and Shieh et al., Fluorogenic Azidofluoresceins for Biological Imaging, J. Am. Chem. Soc. 134(42): 17428-17431 (2012), both of which are incorporated by reference herein in their entirety for all purposes.

As discussed herein, the disclosed assay may involve the use of a multi-well plate or microplate. In some implementations, the plate is pretreated to selectively bind bacteria of interest, thereby improving processing signal to noise and selectivity. A filter plate may also be used for significantly improving processing speed and efficiency. Such implementations may be useful in tests against, by way of example, Tuberculosis, wherein non-pathogenic bacterial contaminants may potentially overwhelm the signal from a slow growing pathogen. In some implementations, multiple antibiotics may be tested on a single plate. Table 3, below, provides an example plate layout for labeling and tagging in accordance with the disclosed methods.

TABLE 3
Example Plate Layout

| | | | HPG (50 μg/mL); | | HPG (50 μg/mL); | | | |
|---|---|---|---|---|---|---|---|---|
| | | | Chloramphenicol | | Nitrofurantoin | | L-Methionine | |
| | HPG (50 μg/mL) | | (100 μg/mL) | | (200 μg/mL) | | (50 μg/mL) | |
| | Test 1 | Test 2 | Test 1 | Test 2 | Test 1 | Test 2 | Test 1 | Test 2 |
| Azide-488 | 0.3 mL | 0.3 mL | 0.3 mL | 0.3 mL | 0.3 mL | 0.3 mL | 0.3 mL | 0.3 mL |
| (50 μM) | culture | culture | culture | culture | culture | culture | culture | culture |
| PBS alone | 0.3 mL | 0.3 mL | 0.3 mL | 0.3 mL | 0.3 mL | 0.3 mL | 0.3 mL | 0.3 mL |
| | culture | culture | culture | culture | culture | culture | culture | culture |

Some implementations of the disclosed assay utilize variations of click chemistry that do not involve copper catalysis such as, for example, the use of a strained azide or strained alkyne that is highly reactive and does not require catalysis. See, for example, Friscourt et al., A Fluorogenic Probe for the Catalyst-Free Detection of Azide-Tagged Molecules, J Am Chem Soc. 134(45): 18809-18815 (Nov. 14, 2012), which is incorporated by reference herein, in its entirety, for all purposes. In addition to ELISA-based detection methods, fluorescent microscopy in a pathology lab is used to provide the advantage of being able to distinguish which bacteria in a mixed culture is defeating the antibiotic. Such complex image-based methods are enabled by using image analysis techniques and artificial intelligence (AI)-based software for determining results.

All literature and similar material cited in this application, including, but not limited to, patents, patent applications, articles, books, treatises, and web pages, regardless of the format of such literature and similar materials, are expressly incorporated by reference in their entirety. Should one or more of the incorporated references and similar materials differs from or contradicts this application, including but not limited to defined terms, term usage, described techniques, or the like, this application controls.

As previously stated and as used herein, the singular forms “a,” “an,” and “the,” refer to both the singular as well as plural, unless the context clearly indicates otherwise. The term “comprising” as used herein is synonymous with “including,” “containing,” or “characterized by,” and is inclusive or open-ended and does not exclude additional, unrecited elements or method steps. Although many methods and materials similar or equivalent to those described herein can be used, particular suitable methods and materials are described herein. Unless context indicates otherwise, the recitations of numerical ranges by endpoints include all numbers subsumed within that range. Furthermore, references to “one implementation” are not intended to be interpreted as excluding the existence of additional implementations that also incorporate the recited features. Moreover, unless explicitly stated to the contrary, implementations “comprising” or “having” an element or a plurality of elements having a particular property may include additional elements whether or not they have that property.

The terms “substantially” and “about”, if or when used throughout this specification describe and account for small fluctuations, such as due to variations in processing. For example, these terms can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%, and/or 0%.

Underlined and/or italicized headings and subheadings are used for convenience only, do not limit the disclosed subject matter, and are not referred to in connection with the interpretation of the description of the disclosed subject matter. All structural and functional equivalents to the elements of the various implementations described throughout this disclosure that are known or later come to be known to those of ordinary skill in the art are expressly incorporated herein by reference and intended to be encompassed by the disclosed subject matter. Moreover, nothing disclosed herein is intended to be dedicated to the public regardless of whether such disclosure is explicitly recited in the above description.

There may be many alternate ways to implement the disclosed technology. Various functions and elements described herein may be partitioned differently from those shown without departing from the scope of the disclosed technology. Generic principles defined herein may be applied to other implementations. Different numbers of a given module or unit may be employed, a different type or types of a given module or unit may be employed, a given module or unit may be added, or a given module or unit may be omitted.

Regarding this disclosure, the term “a plurality of” refers to two or more than two. Unless otherwise clearly defined, orientation or positional relations indicated by terms such as “upper” and “lower” are based on the orientation or positional relations as shown in the Figures, only for facilitating description of the disclosed technology and simplifying the description, rather than indicating or implying that the referred devices or elements must be in a particular orientation or constructed or operated in the particular orientation, and therefore they should not be construed as limiting the disclosed technology. The terms “connected”, “mounted”, “fixed”, etc. should be understood in a broad sense. For example, “connected” may be a fixed connection, a detachable connection, or an integral connection, a direct connection, or an indirect connection through an intermediate medium. For an ordinary skilled in the art, the specific meaning of the above terms in the disclosed technology may be understood according to specific circumstances.

It should be appreciated that all combinations of the foregoing concepts and additional concepts discussed in greater detail herein (provided such concepts are not mutually inconsistent) are contemplated as being part of the disclosed technology. In particular, all combinations of claimed subject matter appearing at the end of this disclosure are contemplated as being part of the technology disclosed herein. While the disclosed technology has been illustrated by the description of example implementations, and while the example implementations have been described in certain detail, there is no intention to restrict or in any way limit the scope of the appended claims to such detail. Additional advantages and modifications will readily appear to those skilled in the art. Therefore, the disclosed technology in its broader aspects is not limited to any of the specific details, representative devices and methods, and/or illustrative examples shown and described. Accordingly, departures may be made from such details without departing from the spirit or scope of the general inventive concept.

What is claimed:

1. A method for determining the susceptibility of a bacteria to an antibiotic, comprising:
(a) transferring one portion of a patient sample containing living bacterial cells into a bacterial growth medium to create a control sample;
(b) transferring another portion of the patient sample into a bacterial growth medium to which a predetermined amount of antibiotic or predetermined amount of a library of antibiotics has been added to create a test sample;
(c) adding an alkyne-modified non-canonical amino acid to the bacterial growth medium of both the control sample and test sample during bacterial growth, wherein the alkyne-modified non-canonical amino acid incorporates into surface proteins, internal proteins, or a combination of surface proteins and internal proteins of the growing bacteria;
(d) reacting the alkyne-containing proteins with an azide-modified detection molecule using click-chemistry to label the living bacteria cells in a detectable manner;
(e) detecting the labeled bacterial cells using a method that generates a detectable signal; and
(f) comparing the signal generated by the control sample to the signal generated by the test sample, wherein a decrease in detectable signal between the control sample and the test sample is indicative of susceptibility of the living bacteria to the predetermined antibiotic or predetermined library of antibiotics.

2. The method of claim 1, wherein the patient sample is a biological sample derived from a bodily fluid or other bodily source.

3. The method of claim 1, wherein the antibiotic is chloramphenicol or any other antibiotic or combination of antibiotics.

4. The method of claim 1, wherein the non-canonical amino acid is azide-modified rather than alkyne-modified and wherein the detection molecule is alkyne-modified rather than azide-modified.

5. The method of claim 1, wherein the alkyne-modified non-canonical amino acid is L-Homopropargylglycine.

6. The method of claim 1, wherein the azide-modified detection molecule is a biotinylated ligand; a fluorogenic azide probe; or a fluorogenic dye. 7 The method of claim 1, wherein the method that generates a detectable signal is fluorescence-based; enzyme-linked immunosorbent assay (ELISA)-based; cell-based, including P5G7 or P2D8 cells; dot blot-based; or microscopy-based.

8. The method of claim 1, wherein the signal is quantifiable, and wherein a predetermined amount of signal is indicative of a minimal inhibitory concentration of antibiotic.

9. The method of claim 1, wherein the method is high-throughput method executed on a multi-well plate or microplate, wherein the type of multi-well plate or microplate includes filter plates, and wherein more than one type of antibiotic may be tested on the multi-well plate or microplate.

10. A method for determining the susceptibility of a bacteria to an antibiotic, comprising:
(a) obtaining a patient sample containing living bacterial cells, wherein the patient sample is a biological sample derived from a bodily fluid or other bodily source;
(b) transferring one portion of the patient sample into a bacterial growth medium to create a control sample;
(c) transferring another portion of the patient sample into a bacterial growth medium to which a predetermined amount of antibiotic or predetermined amount of a library of antibiotics has been added to create a test sample;
(d) adding an alkyne-modified non-canonical amino acid to the bacterial growth medium of both the control sample and test sample during bacterial growth, wherein the alkyne-modified non-canonical amino acid incorporates into surface proteins, internal proteins, or a combination of surface proteins and internal proteins of the growing bacteria, and wherein the alkyne-modified non-canonical amino acid is L-Homopropargylglycine;
(e) reacting the alkyne-containing proteins with an azide-modified detection molecule using click-chemistry to label the living bacteria cells in a detectable manner;
(f) detecting the labeled bacterial cells using a method that generates a detectable signal; and
(g) comparing the signal generated by the control sample to the signal generated by the test sample, wherein a decrease in detectable signal between the control sample and the test sample is indicative of susceptibility of the living bacteria to the predetermined antibiotic or predetermined library of antibiotics.

11. The method of claim 10, wherein the antibiotic is chloramphenicol or any other antibiotic or combination of antibiotics.

12. The method of claim 10, wherein the non-canonical amino acid is azide-modified rather than alkyne-modified and wherein the detection molecule is alkyne-modified rather than azide-modified.

13. The method of claim 10, wherein the azide-modified detection molecule is a biotinylated ligand; a fluorogenic azide probe; or a fluorogenic dye.

14. The method of claim 10, wherein the method that generates a detectable signal is fluorescence-based; enzyme-linked immunosorbent assay (ELISA)-based; cell-based including P5G7 or P2D8 cells; dot blot-based; or microscopy-based.

15. The method of claim 10, wherein the signal is quantifiable, and wherein a predetermined amount of signal is indicative of a minimal inhibitory concentration of antibiotic.

16. The method of claim 10, wherein the method is high-throughput method executed on a multi-well plate or microplate, wherein the type of multi-well plate or microplate includes filter plates, and wherein more than one type of antibiotic may be tested on the multi-well plate or microplate.

17. A method for determining the susceptibility of a bacteria to an antibiotic, comprising:
(a) obtaining a patient sample containing living bacterial cells, wherein the patient sample is a biological sample derived from a bodily fluid or other bodily source;
(b) transferring one portion of the patient sample into a bacterial growth medium to create a control sample;
(c) transferring another portion of the patient sample into a bacterial growth medium to which a predetermined amount of antibiotic or predetermined amount of a library of antibiotics has been added to create a test sample;
(d) adding an alkyne-modified non-canonical amino acid to the bacterial growth medium of both the control sample and test sample during bacterial growth, wherein the alkyne-modified non-canonical amino acid incorporates into surface proteins, internal proteins, or a combination of surface proteins and internal proteins of the growing bacteria, and wherein the alkyne-modified non-canonical amino acid is L-Homopropargylglycine;
(e) reacting the alkyne-containing proteins with an azide-modified detection molecule using click-chemistry to label the living bacteria cells in a detectable manner, wherein the azide-modified detection molecule is a biotinylated ligand; a fluorogenic azide probe; or a fluorogenic dye;
(f) detecting the labeled bacterial cells using a method that generates a detectable signal; and
(g) comparing the signal generated by the control sample to the signal generated by the test sample, wherein a decrease in detectable signal between the control sample and the test sample is indicative of susceptibility of the living bacteria to the predetermined antibiotic or predetermined library of antibiotics.

18. The method of claim 17, wherein the antibiotic is chloramphenicol or any other antibiotic or combination of antibiotics; and wherein the method that generates a detectable signal is fluorescence-based; enzyme-linked immunosorbent assay (ELISA)-based; cell-based including P5G7 or P2D8 cells; dot blot-based; or microscopy-based.

19. The method of claim 17, wherein the non-canonical amino acid is azide-modified rather than alkyne-modified and wherein the detection molecule is alkyne-modified rather than azide-modified.

20. The method of claim 17, wherein the signal is quantifiable, and wherein a predetermined amount of signal is indicative of a minimal inhibitory concentration of antibiotic; wherein the method is high-throughput method executed on a multi-well plate or microplate, wherein the type of multi-well plate or microplate includes filter plates, and wherein more than one type of antibiotic may be tested on the multi-well plate or microplate.
